(19) United States
(12) Patent Application Publication
(10) Pub. No.: US 2024/0018555 A1
(43) Pub. Date: Jan. 18, 2024

(54) UPCYCLING MIXED WASTE PLASTIC THROUGH CHEMICAL DEPOLYMERIZATION AND BIOLOGICAL FUNNELING

(71) Applicant: Alliance for Sustainable Energy, LLC, Golden, CO (US)

(72) Inventors: Gregg Tyler BECKHAM, Golden, CO (US); Lucas Delano ELLIS, Corvallis, OR (US); Kevin P. SULLIVAN, Golden, CO (US); Allison Jean Zimont WERNER, Denver, CO (US)

(21) Appl. No.: 18/256,542

(22) PCT Filed: Dec. 16, 2021
(86) PCT No.: PCT/US21/63725

§ 371 Date: Jun. 8, 2023

Related U.S. Application Data
(60) Provisional application No. 63/126,153, filed on Dec. 16, 2020.

(51) Int. Cl. C12P 7/50; C08J 11/16; C08J 11/10; C08J 11/28

(52) U.S. Cl. CPC C12P 7/50; C08J 11/16; C08J 11/105; C08J 11/28; C08J 2327/08

(57) ABSTRACT

The provided methods and systems describe the breakdown of plastic materials into valuable products, thereby both eliminating waste and providing reusable materials. The described systems and methods utilize catalytic depolymerization and biological funneling via bacteria, which may reduce the costs of recycling plastics in terms of expensive catalysts, energy, and time. Advantageously, some embodiments may target mixed plastic streams, which due to having multiple chemical compositions, may not be easily recycled via conventional recycling techniques. Such mixed plastic streams are currently often discarded (e.g., landfilled) rather than recycled due to the cost and effort required for separating the various compositions present.

### CROSS-REFERENCE TO RELATED APPLICATIONS

This application claims priority from U.S. Provisional Patent Application No. 63/126,153, filed on Dec. 16, 2021, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### SUMMARY

The provided methods and systems describe the breakdown of plastic materials into valuable products, thereby both eliminating waste and providing reusable materials. The described systems and methods utilize catalytic depolymerization and biological funneling via bacteria, which may reduce the costs of recycling plastics in terms of expensive catalysts, energy, and time. Advantageously, some embodiments may target mixed plastic streams, which due to having multiple chemical compositions, may not be easily recycled via conventional recycling techniques. Such mixed plastic streams are currently often discarded (e.g., landfilled) rather than recycled due to the cost and effort required for separating the various compositions present.

Also described herein are novel microorganisms designed to facilitate the chemical decomposition of plastic materials or intermediate materials which have already been partially processed via another recycling method such as catalytic depolymerization. The use of the microorganisms described herein may be advantageous in the processing of plastics by facilitating mixed plastic stream recycling, reducing energy requirements of processing, and reducing costs associated with catalysts.

In an aspect, provided is a method comprising: a) reacting a plastic in the presence of an initiator, a catalyst and a solvent thereby generating an intermediate; catabolizing said intermediate with a non-naturally occurring bacterium thereby generating a product. In some cases, the intermediate may generated without the use of an initiator, which would be beneficial in the reduction of both cost and complexity.

The initiator may comprise a radical initiator, for example, N-hydroxypthalimide (NHPI). The catalyst may comprise a transition metal, for example, Co, Mn, or a combination thereof. For example, a mixture of Co and Mn at a ratio of 5%, 10%, 15%, 20%, or 25% Co to Mn.

The described methods and system may be useful recycling a variety of plastic materials, including polymers and resins. For example, the plastics may comprise polystyrene, polyethylene polyethylene terephthalate (PET), acrylonitrile butadiene styrene (ABS), poly(vinylidene chloride) (PVDC), a polyolefin or any combination thereof.

The intermediate may comprise carboxylic acids or dicarboxylic acids having a number of carbon atoms selected from the range of 7 to 15. Where the plastic comprises PVDC, the intermediate products may comprise a chlorocarboxylic acid.

The solvent may be a polar or a non-polar solvent. The solvent may comprise acetic acid, ethyl acetate, benzene, water, acetonitrile, or a combination thereof. The step of reacting may be performed in the presence of oxygen, including wherein oxygen is considered a reactant. The step of reacting may be performed at a temperature less than or equal to 400° C., 300° C., 250° C., 200° C., 150° C., or optionally, 100° C. The step of reacting may be performed at a pressure less than 200 bar, 150 bar, 100 bar, 80 bar, or optionally, 50 bar.

The bacterium may be of the strain Pseudomonas, for example, a genetically engineered or non-naturally occurring strain of Pseudomonas putida. The product may comprise polymer precursors, for example, polyhydroxyalkanoates (PHAs) or β-ketoadipate. The step of reacting may comprise at least two plastics.

In an aspect, provided is a method for generating polyhydroxyalkanoates (PHAs) or β-ketoadipate comprising: a) reacting a plastic selected from the group of: polystyrene, polyethylene terephthalate (PET), acrylonitrile butadiene styrene (ABS), poly(vinylidene chloride) (PVDC) and a polyolefin; in the presence of a N-hydroxypthalimide (NHPI) initiator, oxygen, a transition metal catalyst, and a solvent thereby generating one or more carboxylic acids, dicarboxylic acids or chloroacetic acids; and b) catabolizing said one or more intermediate products with Pseudomonas putida bacteria thereby generating polyhydroxyalkanoates (PHAs) or.

The step of reacting may comprise at least two plastics selected from the group of: polystyrene, polyethylene terephthalate (PET), acrylonitrile butadiene styrene (ABS), poly(vinylidene chloride) (PVDC) and a polyolefin. The transition metal catalyst may be Co, Mn, or a combination thereof.

In an aspect, provided is a system for performing any of the methods described herein.

In an aspect, provided is a non-naturally occurring Pseudomonas capable of producing polyhydroxyalkanoates, wherein said Pseudomonas is capable of catabolizing terephthalate, glycolate, benzoate, adipate or C₄-C₁₇ dicarboxylates. The Pseudomonas may be capable of catabolizing terephthalate, glycolate and adipate.

The Pseudomonas may further comprise an exogenous gene from a Comamonas, for example, a gene that encodes for tphA1, tphA2, tphA3 and/or tphB. The Pseudomonas may further comprise an exogenous gene from a Rhodococcus jostii, for example, a gene that encodes for RHA1 and/or tpak. The Pseudomonas may further comprise an exogenous gene from a Acenitobacter baylyi, for example, a gene that encodes for ADP1, dcaA, dcaI, dcaK, dcaJ and/or dcaP. The Pseudomonas may be P. putida KT2440. The Pseudomonas may have the gene psrA deleted.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates a hybrid catalytic depolymerization of plastics and biological funneling to useful products.

FIG. 2 illustrates a process for oxidative depolymerization of plastic waste. FIG. 2A Plastic types and their corresponding oxidative depolymerization products. FIG. 2B Results of oxidation of PS (Mw=250 kDa) using the Co/Mn/NHPI/O₂ system. Conditions: 500 mg PS, 50 mg (10 mol %) NHPI, 0.5-20 mg (0.1-4 mol %) Co(OAc)₂, 5-20 mg (1-4 mol %) Mn(OAc)₂, AA (acetic acid) or AA:EA (1:1 v/v acetic acid: ethyl acetate), 180° C., 8 bar O₂ in 72 bar inert, and 5 hour reaction duration. Products analyzed by HPLC using a diode array detector, and yields are reported relative to the total aromatic content of the PS. FIG. 2C Chromatogram from LC-MS for the reaction products from the oxidation of PE with the Co/Mn/NHPI/O₂ system. Conditions: 500 mg PE (Mw=60 kDa), 50 mg (10 mol %) NHPI, 0.5-20 mg (0.1-4 mol %) Co(OAc)₂, 5-20 mg (1-4 mol %) Mn(OAc)₂, 30 mL AA:EA (1:1 v/v acetic acid: ethyl acetate), 180° C., 8 bar I₂ in 72 bar inert, and 15 hour reaction duration. C7-C15 represent dicarboxylic acids with the indicated carbon chain length.

FIG. 3 illustrates catabolism of mixed stream for catalysis effluent by engineered P. putida. FIG. 3A Model compounds predicted to result from the oxidative catalysis of polystyrene (PS), polyethylene terephthalate (PET), polypropylene (PP), and polyethylene (PE). Engineering modifications are indicated in boxes where superscripts following the gene name indicates the host organism (E6, Comomonas sp. E6; RHA1, Rhodococcus jostii RHA1; and ADP1, Acinetobacter baylyi ADP1). FIG. 3B Growth of wild-type P. putida or TDM461 (P. putida KT2440 ΔPP_4740-4741::P_{tac}:tphA2_{II}A3_{II}B_{II}Al_{II}^{E6} P_{tac}:tpaK^{RHA1}) in M9 minimal media supplemented with 100 mM TPA. FIG. 3C Growth of LJ164 (P. putida KT2440 ΔgclR) and RC024 (P. putida KT2440 ΔgclR P_{tac}:glcDEFG:PP_3749) in M9 minimal supplemented with 100 mM ethylene glycol, a precursor to glycolate. FIG. 3D Growth of RC026 (P. putida KT2440 ΔPP_4740-4741::P_{tac}:tphA2_{II}A3_{II}B_{II}Al_{II}^{E6} P_{tac}:tpaK^{RHA1} ΔglcR P_{tac}:glcDEFG:PP_3749) and AW061 (P. putida KT2440 ΔPP_4740-4741::P_{tac}:tphA2_{II}A3_{II}B_{II}Al_{II}^{E6} P_{tac}:tpaK^{RHA1} ΔglcR P_{tac}:glcDEFG:PP_3749 ΔpaaX::P_{tac}:dcaAKIJP^{ADP1}) in M9 minimal media supplemented with 5 mM or 15 mM adipate, respectively. FIG. 3E Growth of AW061 in M9 minimal media supplemented with 15 mM acetate and 5 mM of benzoate, terephthalate, acetoxyacetate, glycolate, formate, succinate, glutarate, and adipate, each. FIG. 3F Growth of AW061 in the conditions listed for FIG. 3E but with the addition of 1 mM N-Hydroxyphthalimide, cobalt-acetate, and manganese-acetate, each. All cultivations were performed in M9 minimal media in a BioscreenC® held at 30° C. and shaking at maximum speed. Error bars represent the standard deviation across biological triplicates.

FIG. 4 illustrates autoxidation process for PVDC films based on TGA analysis.

### DETAILED DESCRIPTION

The embodiments described herein should not necessarily be construed as limited to addressing any of the particular problems or deficiencies discussed herein. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

As used herein, the term “Chlorocarboxylic acid” refers to a molecule that contains at least one C1 atom and at least one carboxylic acid functional group, for example, chloroacetic acid.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

### EXAMPLE 1

### Oxidative Funneling

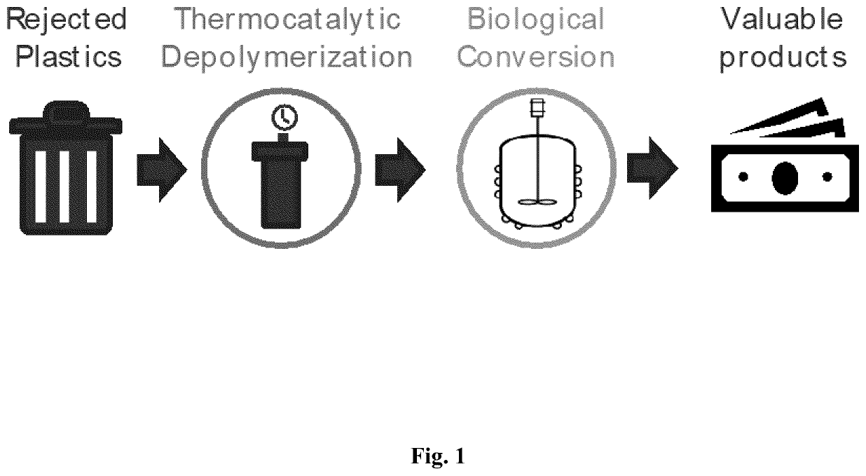

Described herein is a hybrid process wherein mixed, rejected, post-consumer plastics are thermocatalytically depolymerized into a heterogeneous mixture of compounds, which are then biologically converted into a single valuable product (FIG. 1). Details and data demonstrating functionality are provided for each of the two main processes.

### Part I—Thermocatalytic Pretreatment

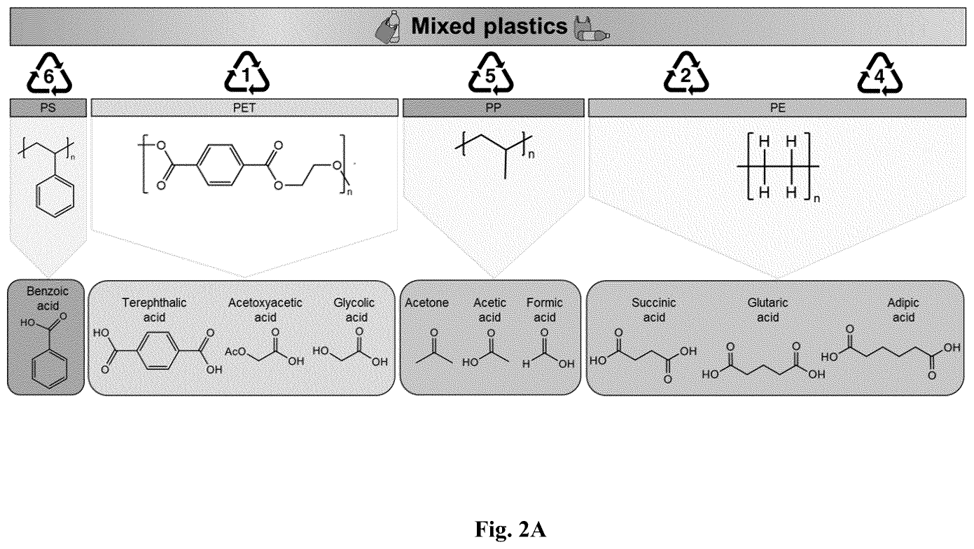

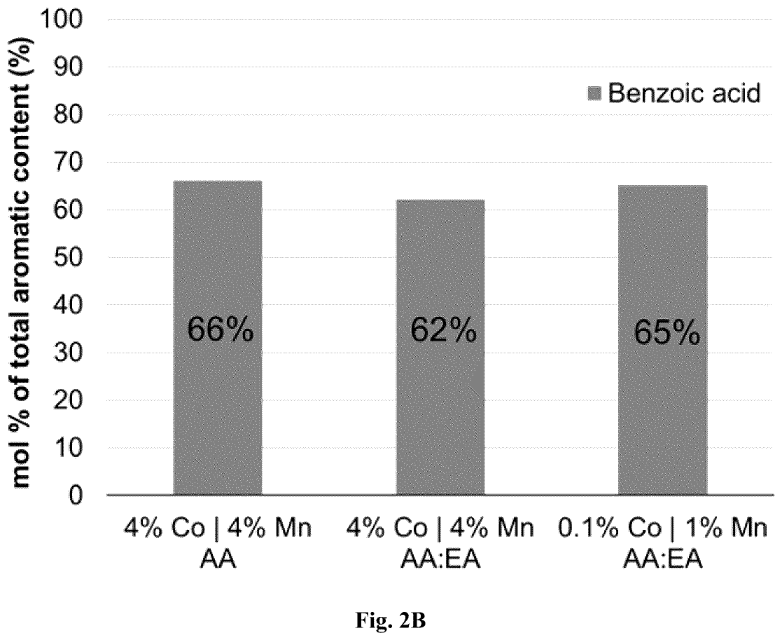

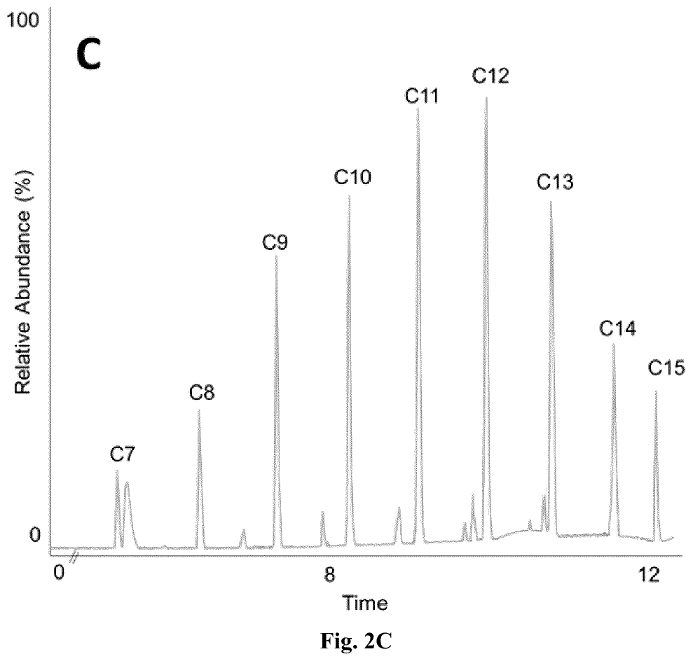

Catalytic depolymerization is used to convert plastic feedstocks into small-molecule products that can be catabolized by microbial organisms. Described is a method that uses a radical-based pathway with dioxygen (e.g., air), radical initiators, and catalysts, that ultimately results in cleavage of C—C bonds in the backbones of a variety of polymers (FIG. 2A) and formation of small molecule carboxylic acids. This demonstrates that a Co/Mn/NHPI/O₂ system can cleave the C—C bonds of polystyrene and polyethylene substrates to generate small-molecule organic acids and achieve a 65% yield of benzoic acid from polystyrene (FIG. 2B). Parameters that affect the outcome of this complex catalytic system include the ratio of metal species, concentration radical initiator, and O₂ pressure. For example, a ratio of 10% Co relative to Mn results in higher activity than equimolar amounts, even when higher metal concentrations are used in the equimolar system (FIG. 2B). Model compound studies have revealed several details about the system, including the complex role of the concentration of the initiator species, where increased mol % of NHPI results in higher conversions of the reactant, but lower selectivity toward the desired product. One result is a significant increase in conversion as the O₂ pressure is increased, suggesting that the oxygen concentration is likely a limiting factor in this system. Dissolution of the plastic also plays a role in the process. Also described are several solvents and combinations of solvents to facilitate solubilization of the polymer, including acetic acid, ethyl acetate, benzene, water, and acetonitrile.

Described are examples using this catalytic system on PE substrates, resulting in a mixture of dicarboxylic acids products of various carbon length, as shown in FIG. 2C. One challenge with this system is selectivity toward biologically relevant products, in other words, the ability to control the dicarboxylic acid distribution to obtain short chain dicarboxylates that are able to be catabolized in the downstream biological processing. Based on results from PS systems, the optimization of the tandem catalyst system (i.e., initiator and metals) or various reaction engineering controls, like increasing O₂ pressure, will increase selectivity towards small products. Together, these results indicate success of this catalytic system to cleave two different polyolefin polymers to small molecule compounds. Simultaneous optimization of the tandem catalyst system and process engineering, with a focus on biocompatibility and TEA, may describe a process capable of handling the heterogeneous mixed plastic streams of today's waste into a feedstock for subsequent biological upcycling.

### Part II—Biological Funneling of Mixed Plastic Products

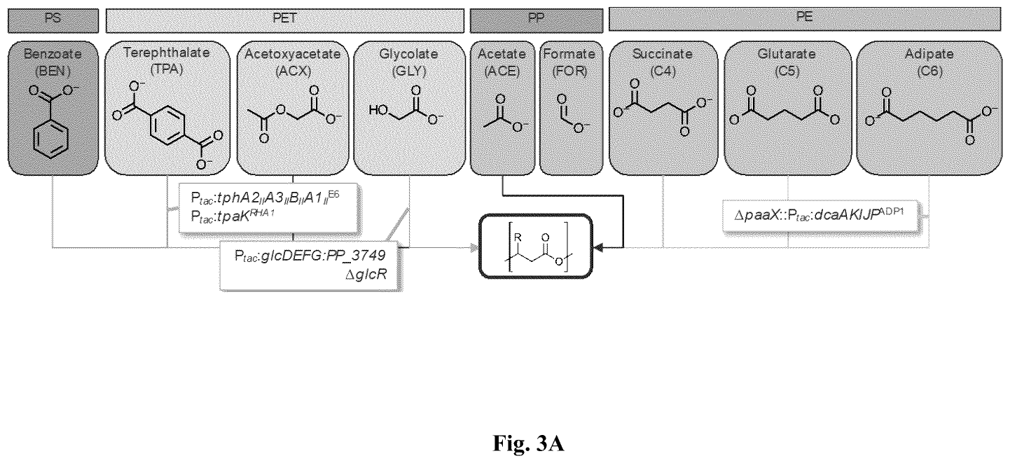

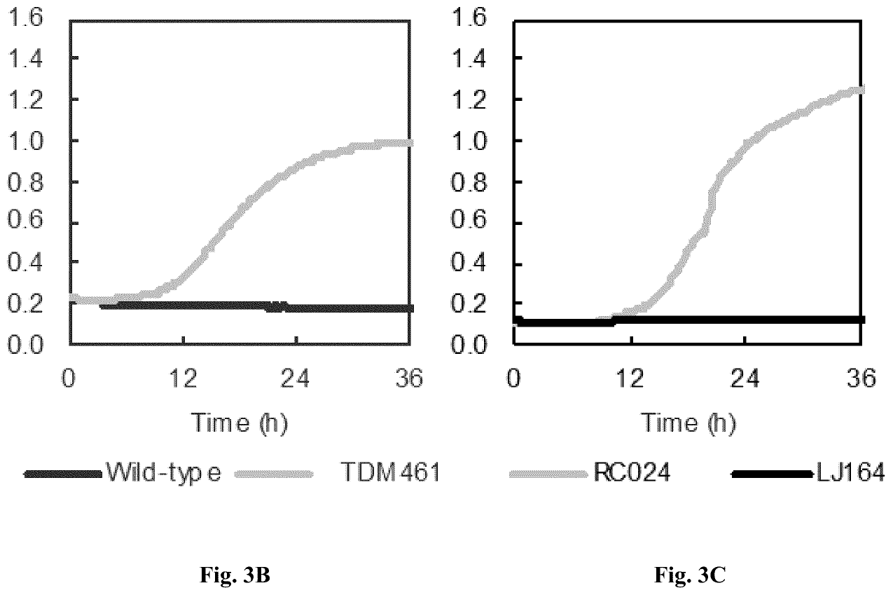

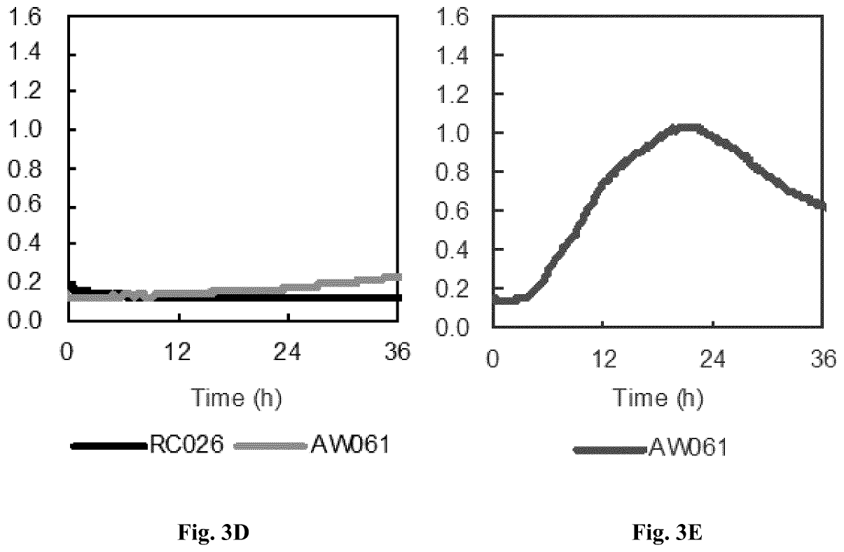

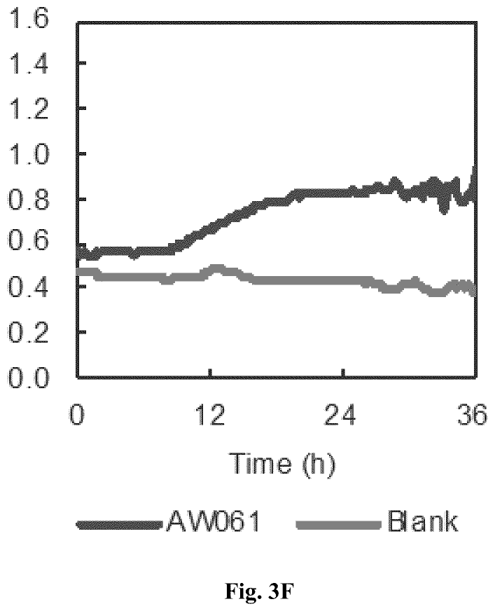

Also described is a strain of Pseudomonas putida KT2440 (hereafter P. putida) to catabolize all of the major products in the catalysis effluent (FIG. 3A). P. putida is a metabolically versatile and robust Gram-negative bacterium that has been successfully employed for the valorization of heterogeneous lignin steams into polyhydroxyalkanoates (PHAs) via biological funneling. An analogous approach was taken here with the main differences being that many of the predicted products for biological funneling of plastics are not native substrates. Benzoate, formate, succinate, and glutarate are utilized by wild-type P. putida. Described is an engineered P. putida to catabolize the three the major catalysis products into PHA products that do not support growth of wild-type P. putida: terephthalate (TPA), glycolate (GLY), and adipate (C6). To engineer catabolism of TPA, the tphA2_{II}A3_{II}B_{II}Al_{II} TPA catabolic operon from Comamonas sp. E6 is integrated into the PP_4740-4741genomic locus and the putative tpaK TPA transporter from Rhodococcus jostii RHA1 is integrated into the fpvA genomic locus, both driven by the constitutive and strong promoter P_{tac}. These modifications enable growth on 100 mM TPA (FIG. 3B). To enable robust catabolism of glycolate, the native glcDEFG:PP_3749 operon is overexpressed in addition to deletion of the gclR regulator, these modifications improve ethylate glycol catabolism (a precursor to glycolate). The combination of these modifications enable growth on 100 mM ethylene glycol (FIG. 3C). To engineer adipate catabolism, the dcaAKIJP operon from Acenitobacter baylyi ADP1 is integrated into the chromosome with simultaneous deletion of paaX. PaaX is a putative repressor the paa operon. Based on characterization of phenylacetate catabolism in P. putida, paaFHJ may be analogous to dcaEHF in A. baylyi and together with the dca engineering enables catabolism of adipate. This results in poor growth in media with adipate as the sole carbon source (FIG. 3D) but may be improved. Nonetheless, the resulting strain with all of these genetic modifications grows in a mixture of all compounds (FIG. 3E) even with 1 mM of the NHPI initiator and catalysts cobalt acetate and manganese acetate (FIG. 3F). NHPI rapidly precipitates out of the media in water making spectrophotometric measurements of cell growth difficult at concentrations above 1 mM. A strain that rapidly catabolizes all compounds in the catalysis effluent, PHA production will be induced as described herein.

### EXAMPLE 2

### Autooxidation of PVDC and PE films

Poly(vinylidene chloride) (PVDC) is often combined with polyethylene (PE) in industrial packaging materials. Because PVDC is prone to thermal de-chlorination and crosslinking at elevated temperatures (T˜250° C.), PVDC-containing plastics are commonly unable to be recycled. To overcome this problem, we describe an autoxidation processes to enable simultaneous chemical recycling of PVDC and PE.

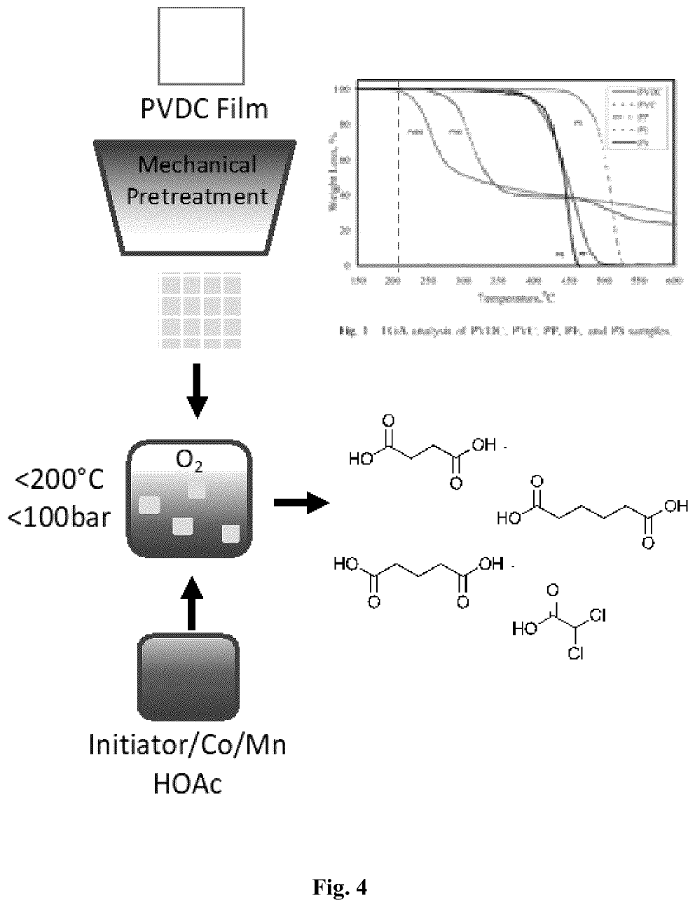

To address the challenge of reprocessing PVDC-containing plastics while avoiding de-chlorination and crosslinking, mild oxidative catalysis is employed to deconstruct both the polyolefin and PVDC components simultaneously, generating a mixture of processable carboxylic acid intermediates (FIG. 4). This approach utilizes oxygen (in air) as the oxidant, a radical initiator (such as N-hydroxyphthalimide), and transition metal catalysts (such as Co(II) and Mn(II)) to guide reaction selectivity towards C—C cleavage in a homogeneous system at moderate temperatures and pressures (T<200° C., P<10² bar). Acetic acid is used as a solvent. This process yields dicarboxylic acids with tunable carbon chain lengths from polyolefins, while the PVDC yields chloroacetic acids. This process with optimized reaction conditions maximizes yield of the desired products, while avoiding the generation of HCl and cross-linked products.

The invention may be further understood by the following non-limiting examples:

1. A method comprising:
reacting a plastic in the presence of a catalyst and a solvent thereby generating an intermediate;
catabolizing the intermediate with a non-naturally occurring bacterium thereby generating a product.
   2. The method of example 1, wherein the step of reacting further comprising an initiator, wherein the initiator is a radical.
   3. The method of example 2, wherein the radical initiator comprises N-hydroxypthalimide (NHPI).
   4. The method of any of examples 1-3, wherein the catalyst comprises a transition metal.
   5. The method of any of examples 1-4, wherein the catalyst comprises Co, Mn, or a combination thereof.
   6. The method of any of examples 1-5, wherein the plastic comprises polystyrene, polyethylene, polyethylene terephthalate (PET), acrylonitrile butadiene styrene (ABS), poly(vinylidene chloride) (PVDC), a polyolefin or any combination thereof.
   7. The method of any of examples 1-6, wherein the intermediate comprises at least one of a carboxylic acid or dicarboxylic acid having a number of carbon atoms selected from the range of 4 to 22.
   8. The method of any of examples 1-6, wherein the plastic is PVDC and the intermediate comprises a chlorocarboxylic acid.
   9. The method of any of examples 1-8, wherein the solvent comprises acetic acid, ethyl acetate, benzene, water, acetonitrile, or a combination thereof.
   10. The method of any of examples 1-9, wherein the step of reacting is performed in the presence of oxygen.
   11. The method of any of examples 1-10, wherein the step of reacting is performed at a temperature less than or equal to 200° C.
   12. The method of any of examples 1-11, wherein the step of reacting is performed at a pressure less than or equal to 100 bar.
   13. The method of any of examples 1-12, wherein the bacterium is of the strain Pseudomonas.
   14. The method of any of examples 1-13, wherein the bacterium is a genetically engineered strain of Pseudomonas putida.
   15. The method of example 13 or 14, wherein the bacterium has the genes pcal and pcaJ deleted.
   16. The method of any of examples 1-15, wherein the product comprises a polyhydroxyalkanoate (PHA) or β-ketoadipate.
   17. The method of any of examples 1-16, wherein the step of reacting comprises at least two plastics.
   18. A method for generating β-ketoadipate comprising:
reacting a plastic selected from the group of: polystyrene, polyethylene, polyethylene terephthalate (PET), acrylonitrile butadiene styrene (ABS), poly(vinylidene chloride) (PVDC) and a polyolefin; in the presence of oxygen, a transition metal catalyst, and a solvent thereby generating one or more carboxylic acids, dicarboxylic acids or chloroacetic acids;
catabolizing the one or more intermediate products with a non-naturally occurring Pseudomonas putida bacteria thereby generating polyhydroxyalkanoates (PHAs) or β-ketoadipate.
   19. The method of example 18, wherein the step of reacting comprises at least two plastics selected from the group of: polystyrene, polyethylene, polyethylene terephthalate (PET), acrylonitrile butadiene styrene (ABS), poly(vinylidene chloride) (PVDC) and a polyolefin.
   20. The method of example 18 or 19, wherein the transition metal catalyst comprises Co, Mn, or a combination thereof.
   21. The method of any of examples 18-20, wherein the step of reacting the plastic is performed in the presence of an initiator.
   22. The method of example 2 or 21, wherein the initiator is N-hydroxypthalimide (NHPI). 23. A system for performing the methods of any of examples 1-20.
   24. A non-naturally occurring Pseudomonas capable of producing polyhydroxyalkanoates, wherein the Pseudomonas is capable of catabolizing terephthalate, benzoate, adipate or C₄-C₁₇ dicarboxylates.
   25. The Pseudomonas of example 24, wherein the Pseudomonas is capable of catabolizing terephthalate, glycolate and adipate or C₄-C₁₇ dicarboxylates.
   26. The Pseudomonas of example 24 or 25, wherein the Pseudomonas further comprises an exogenous gene from a Comamonas.
   27. The Pseudomonas of example 26, wherein the exogenous gene encodes for tphA1, tphA2, tphA3 and/or tphB.
   28. The Pseudomonas of any of examples 24-27, wherein the Pseudomonas further comprises an exogenous gene from a Rhodococcus jostii.
   29. The Pseudomonas of examples 28, wherein the exogenous gene encodes for RHA1 or tpak.
   The Pseudomonas of any of examples 24-29, wherein the Pseudomonas further comprises an exogenous gene from a Acenitobacter baylyi.
   31. The Pseudomonas of example 30, wherein the exogenous gene encodes for ADP1, dcaA, dcaI, dcaK, dcaJ and/or dcaP.
   32. The Pseudomonas of any of examples 24-31, wherein the Pseudomonas has the gene psrA deleted.
   33. The Pseudomonas of any of examples 24-32, wherein the Pseudomonas is P. putida KT2440.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods, and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges, and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
reacting a plastic in the presence of a catalyst and a solvent thereby generating an intermediate;
catabolizing the intermediate with a non-naturally occurring bacterium thereby generating a product.

2. The method of claim 1, wherein the step of reacting is performed in the presence of an initiator and wherein the initiator comprises a radical initiator.

3. The method of claim 2, wherein the radical initiator comprises N-hydroxypthalimide (NHPI).

4. The method of claim 1, wherein the catalyst comprises Co, Mn, or a combination thereof.

5. The method of claim 1, wherein the plastic comprises polystyrene, polyethylene, polyethylene terephthalate (PET), acrylonitrile butadiene styrene (ABS), poly(vinylidene chloride) (PVDC), a polyolefin or any combination thereof.

6. The method of claim 1, wherein the intermediate comprises at least one of a carboxylic acid or dicarboxylic acid having a number of carbon atoms selected from the range of 4 to 22.

7. The method of claim 1, wherein the plastic is PVDC and the intermediate comprises a chlorocarboxylic acid.

8. The method of claim 1, wherein the solvent comprises acetic acid, ethyl acetate, benzene, water, acetonitrile, or a combination thereof.

9. The method of claim 1, wherein the step of reacting is performed in the presence of oxygen.

10. The method of claim 1, wherein the bacterium is of the strain Pseudomonas.

11. The method of claim 1, wherein the bacterium is a genetically engineered strain of Pseudomonas putida.

12. The method of claim 11, wherein the bacterium has the genes pcal and pcaJ deleted.

13. The method of claim 1, wherein the product comprises β-ketoadipate.

14. A method for generating β-ketoadipate comprising:
reacting a plastic selected from the group of: polystyrene, polyethylene, polyethylene terephthalate (PET), acrylonitrile butadiene styrene (ABS), poly(vinylidene chloride) (PVDC) and a polyolefin; in the presence of oxygen, a transition metal catalyst, and a solvent thereby generating one or more carboxylic acids, dicarboxylic acids or chloroacetic acids;
catabolizing the one or more intermediate products with a non-naturally occurring Pseudomonas putida bacteria thereby generating β-ketoadipate.

15. The method of claim 14, wherein the step of reacting is performed in the presence of a N-hydroxypthalimide (NHPI) initiator.

16. A non-naturally occurring Pseudomonas capable of producing polyhydroxyalkanoates, wherein the Pseudomonas is capable of catabolizing terephthalate, benzoate, adipate or C₄-C₁₇ dicarboxylates.

17. The Pseudomonas of claim 16, wherein the Pseudomonas further comprises an exogenous gene from a Comamonas and wherein the exogenous gene encodes for tphA1, tphA2, tphA3 and/or tphB.

18. The Pseudomonas of claim 16, wherein the Pseudomonas further comprises an exogenous gene from a Rhodococcus jostii and wherein the exogenous gene encodes for tpak.

19. The Pseudomonas of claim 16, wherein the Pseudomonas further comprises an exogenous gene from a Acenitobacter baylyi and wherein the exogenous gene encodes for dcaA, dcaI, dcaK, dcaJ and/or dcaP.

20. The Pseudomonas of claim 16, wherein the Pseudomonas has the gene psrA deleted.
